(19) United States
(12) Patent Application Publication
(10) Pub. No.: US 2019/0239905 A1
(43) Pub. Date: Aug. 8, 2019

(54) RETRACTION DEVICE, SYSTEM, AND METHOD

(71) Applicant: NexGen Medical Systems, Inc., Wayzata, MN (US)

(72) Inventors: Matthew Jonathan Olson, Grafton, ND (US); Keng Thao, St. Paul, MN (US); Patrick Raymond Haley, Elk River, MN (US)

(73) Assignee: NexGen Medical Systems, Inc., Wayzata, MN (US)

(21) Appl. No.: 15/892,139

(22) Filed: Feb. 8, 2018

(51) Int. Cl. A61B 17/221

(52) U.S. Cl. CPC A61B 17/221; A61B 2017/00862

(57) ABSTRACT

Medical systems and methods for making and using medical systems are disclosed. Example medical systems may include devices capable of capturing and facilitating removal of an obstruction in a vessel or cavity. The medical devices may include a shaft having a plurality of eyelets spaced along the shaft. A pull wire may be connected to the shaft and extend through one or more of the plurality of eyelets. Retraction of the pull wire may result in forming one or more loops in the shaft. A core wire may extend along the shaft to provide a desired stiffness at one or more locations along the shaft and/or along a loop formed with the shaft. The shaft may be formed of a coiled wire.

### TECHNICAL FIELD

The present disclosure pertains to medical devices, and methods for manufacturing and using medical devices. More particularly, the present disclosure pertains to medical devices, methods, and systems, for isolating, capturing, and/or removing obstructions from vessels, ducts, and/or cavities of a body.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### BRIEF SUMMARY

This disclosure provides, design, material, manufacturing method, and use alternatives for medical devices and systems. In a first aspect, a retraction device is disclosed that may include a tubular member, a plurality of eyelets spaced along the tubular member, a first elongated member, and a second elongated member. The first elongated member may extend through a first eyelet of the plurality of eyelets. The tubular member may have a first end portion, a second end portion, and a lumen extending from the first end portion to the second end portion, where the second elongated member may extend through the lumen of the tubular member. A stiffness of the second elongated member may vary along a length of the second elongated member.

In a further aspect, a retraction device may include a shaft and an elongated member. The shaft may have a first end portion and a second end portion, where the elongated member may extend along the shaft and connect to the second end portion of the shaft. Retraction of the elongated member toward the first end portion of the shaft may result in forming a first section of a plurality of loops and a second section of a plurality of loops in the shaft. Two or more of the plurality of loops of the first section may have a first diameter that is less than a second diameter of two or more of the plurality of loops of the second section.

In a further aspect, a method of using a retraction device may include retracting a pull wire having a distal end portion connected to a distal end portion of a shaft. Two or more loops of a first set of loops in the shaft and two or more loops of a second set of loops in the shaft may be formed in response to retracting the pull wire. The shaft may be configured such that at least two loops of the first set of loops each has a first diameter and at least two loops of the second set of loops each has a second diameter different than the first diameter.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic side view of an example retraction device;

FIG. 2 is a schematic top view of an example retraction device in a retracted position;

FIG. 3 is a schematic cross-section view of a portion of an example distal shaft of a retraction device;

FIGS. 4A and 4B are schematic cross-section views of portions of an example distal shaft and handle of a retraction device; and

FIGS. 5A-5H are views of a retraction device system schematically showing an example method of using the retraction device system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

The term “diameter”, as used in this specification and the appended claims, is generally employed in its sense as being a line passing from side to side of an object unless the content clearly dictates otherwise. In some cases, the diameter of an object may pass through a center of the object and/or may be a longest line passing from side to side of the object.

As used in this specification and the appended claims, the singular forms “a”, “an”, and “the” include plural referents unless the content clearly dictates otherwise. As used in this specification and the appended claims, and although the term “and/or” is sometimes expressly recited herein, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Cardiovascular disease and peripheral arterial disease may arise from accumulation of atheromatous material on the inner walls of vascular lumens, resulting in a condition known as atherosclerosis. If a partially or completely occluded vessel provides blood to sensitive tissue such as the brain or heart, for example, serious tissue damage may result. Atheromatous and other vascular deposits may restrict blood flow through an artery and can cause ischemia in a heart of a patient, vasculature of a patient's legs, vasculature of a patient's lungs, a patient's carotid artery, etc. Such ischemia may lead to pain, swelling, wounds that will not heal, amputation, stroke, myocardial infarction, and/or other conditions.

One or more conditions or diseases may arise from accumulation of deposits on the inner walls of venous lumens, such as deep vein thrombosis, a pulmonary embolism, venous insufficiency, and/or other conditions or diseases. For example, blood clots or other build-ups of deposits in a vein may result in deep vein thrombosis, which may lead to venous insufficiency, a pulmonary embolism, and/or other diseases or conditions. Symptoms of restricted blood flow through veins due to build ups of deposits and/or other objects may include swelling of legs or ankles, leg cramps, varicose veins, leg ulcers, weak legs, restlessness, lightheadedness, etc.

Vascular deposits or objects in a body, may have widely varying properties, with some deposits or objects being relatively soft and others being fibrous and/or calcified. One example of a deposit or object in a vessel or cavity of a body is a thrombus. A process of forming a thrombus is called thrombosis and this process may produce a clot in a patient's vasculature. Such clots may occasionally be harmlessly dissolved in the blood stream passing through the vasculature. At other times, such cots may lodge in a blood vessel or embolize to a distal blood vessel where they can partially or completely occlude a flow of blood.

Deposits and/or other objects in a body may be treated in a variety of ways including, but not limited to, drugs, bypass surgery, atherectomy, and/or a variety of catheter-based approaches that may rely on intravascular widening or removal of the deposit or other object at least partially occluding a blood vessel or cavity of the body. As described herein, a catheter-based approach may be utilized for removing a thrombus or other deposit and/or object in a vessel or cavity of a body. In the catheter-based approach, an elongated member may be extended past (e.g., distal of) at least a portion a thrombus or other object in a vessel or cavity of a body, a withdrawal member may be formed at a location past at least a portion of the thrombus or other object, and the withdrawal member may be retracted through the vessel or cavity of the body, where retraction of the withdrawal member removes (e.g., withdraws) at least part of the thrombus or other object from the vessel or cavity of the body. The catheter-based approach may utilize a retraction device, as described herein, to facilitate removal of a thrombus or other object from a vessel and/or body cavity.

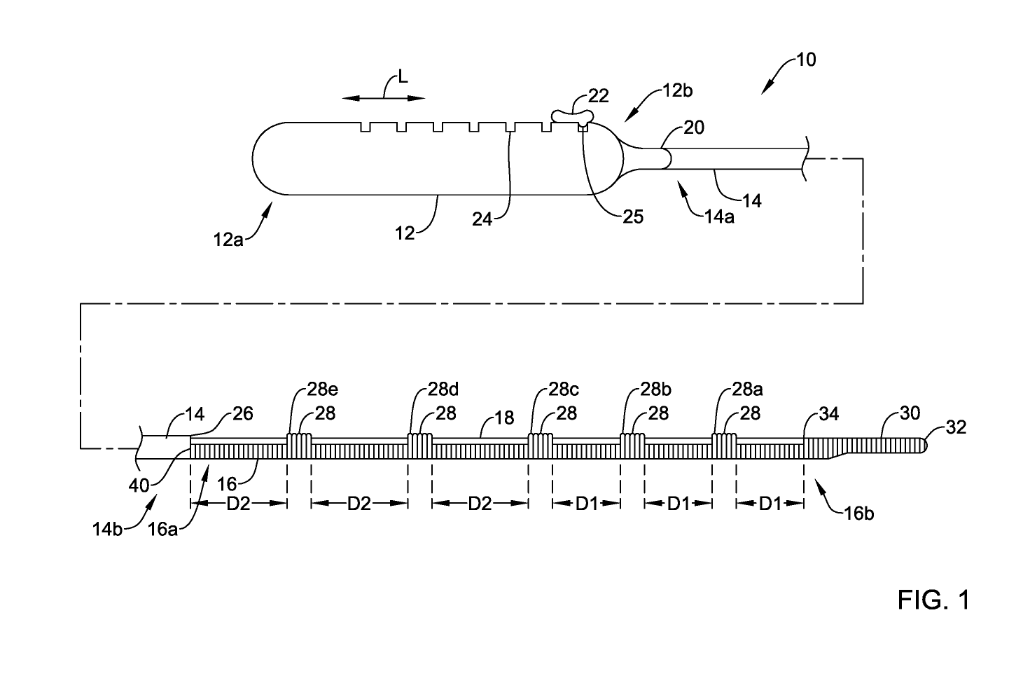

FIG. 1 depicts a schematic side view of a retraction device 10. The retraction device 10 may include a variety of components including, but not limited to, a handle 12, a proximal shaft 14, a distal shaft 16, and a pull wire 18 (e.g., an elongated member). As depicted in FIG. 1, the proximal shaft 14 may extend from a distal portion of the handle 12 and the distal shaft 16 may extend from a distal portion of the proximal shaft 14. In some cases, the proximal shaft 14 may be omitted and the distal shaft 16 may extend distally from the distal portion of the handle 12. Alternatively, the distal shaft 16 may be omitted and the proximal shaft 14 may extend to a distal end of the retraction device 10.

The handle 12 may be any suitable type of handle having a proximal end portion 12a and a distal end portion 12b. The distal end portion 12b of the handle 12 may receive the proximal shaft 14. In some cases, the distal end portion 12b of the handle 12 may be connected or attached to the proximal shaft 14, but this is not required and the handle 12 and the proximal shaft 14 may be connected and/or in communication at one or more other location of the handle 12. In some cases, an adaptor 20 may facilitate a connection and/or movement between the handle 12 and the proximal shaft 14, however, the adaptor 20 may be omitted, as desired.

The handle 12 and/or components thereof, may take on any suitable shape or form. As shown in the Figures, the handle 12 may have an elongated shape with a rounded cross-section, but this is not required and the handle 12 may have one or more suitable additional and/or alternative shapes or forms.

The handle 12 may be made out of a suitable material. In some cases, the handle 12 may be formed from a polymer material, a metal material, a combination of a metal material and a polymer material, and/or one or more other suitable materials. Example polymer and metal materials, among other possible materials, are discussed below. Further, the handle 12 may be formed with a suitable forming technique including, but not limited, to machining, molding, grinding, injection molding, laser cutting, etc.

An adjustment member 22 may be located adjacent the handle 12. The adjustment member 22 may be in communication with the pull wire 18, such that movement (e.g., adjustment) of the adjustment member 22 may cause movement (e.g., adjustment) of the pull wire 18. In one example, movement of the adjustment member 22 may result in retraction of the pull wire 18 through at least a portion of the proximal shaft 14 and/or the distal shaft 16 to form one or more loops in the distal shaft 16 (e.g., as discussed below). In some cases, the handle 12 may include indicia for, among other purposes, indicating a distance the pull wire 18 has been retracted in response to movement of the adjustment member 22.

The adjustment member 22 may be configured to move in one or more directions such that movement of the adjustment member 22 in at least one direction is configured to adjust or move the pull wire 18. In one example, as shown in the Figures, the adjustment member 22 may be configured to adjust in a longitudinal direction, L, such that movement of the adjustment member 22 in the longitudinal direction, L, results in adjustment or movement of the pull wire 18 in the longitudinal direction, L. Alternatively or in addition, the adjustment member 22 may be configured to rotate and/or move in a different suitable direction. In some cases, when the adjustment member 22 is configured to rotate, rotation of the adjustment member 22 may result in linear movement of the pull wire 18 (e.g., movement of the pull wire 18 in the longitudinal direction).

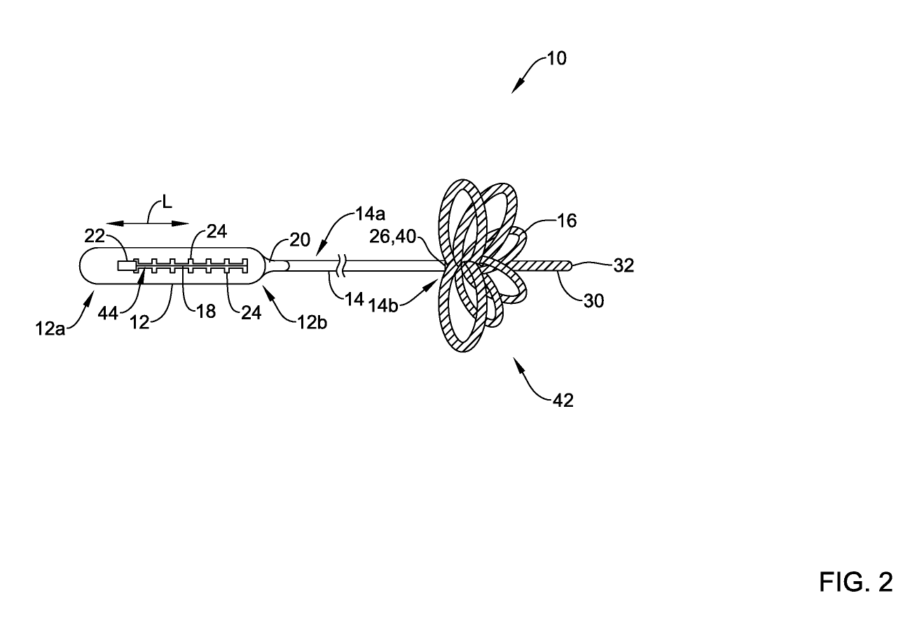

The handle 12 may include and/or define one or more restrictions 24 (e.g., adjustment restrictions), but this is not required in all cases. The one or more restrictions 24 may be formed on and/or in the handle 12 and may be configured to engage the adjustment member 22 as the adjustment member 22 is adjusted or moved. In some cases, two or more of the restrictions 24 may be spaced a predetermined distance from one another and engagement of the adjustment member 22 with a restriction 24 may be indicative of the pull wire 18 being withdrawn a predetermined linear distance since the adjustment member 22 engaged an immediately adjacent restriction 24. Example restrictions 24 may include, but are not limited to, detents, cut-outs, recesses, spacings, notches, indents, bumps, protrusions and/or other features configured to engage the adjustment member 22. As shown in the example of FIGS. 1 and 2, the restrictions 24 may be linearly spaced from one another such that the adjustment member 22 may engage a restriction 24 as it is moved one or more predetermined distances in the longitudinal direction, L. In situations when the adjustment member 22 may be configured to rotate to linearly adjust the pull wire 18, the restrictions 24 may be radially spaced from one another and configured to engage the adjustment member 22 as the adjustment member 22 rotates.

The adjustment member 22 may include a portion 25 configured to engage the restrictions 24 in or on the handle 12 as the adjustment member 22 is moved relative to the handle 12. In one example, the adjustment member 22 may include a portion 25 having a protrusion that is configured to engage the restrictions 24. Alternatively or in addition, the portion 25 of the adjustment member 22 may include, but is not limited to, a detent, cut-out, recess, spacing, notch, indent, and/or other formations to facilitate engaging the restrictions in or on the handle 12. In some cases, the restrictions 24 and/or the portion 25 configured to engage the restrictions 24 may be omitted from the retraction device 10.

The proximal shaft 14, when included in the retraction device 10, may have a proximal end portion 14a, a distal end portion 14b, and one or more lumens extending between the proximal end portion 14a and the distal end portion 14b of the proximal shaft 14. The proximal end portion 14a of the proximal shaft may be directly or indirectly (e.g., through an intermediary such as the adaptor 20 or other intermediary) attached to the handle 12 (e.g., the distal end portion 12b of the handle 12 or other suitable portion of the handle 12). The one or more lumens of the proximal shaft 14 may include a lumen configured to receive the pull wire 18, such that the pull wire 18 may extend from the handle 12 (e.g., from a location within the handle 12 and in communication with the adjustment member 22) through the proximal end portion 14a of the proximal shaft 14, through the lumen of the proximal shaft, and out of the distal end portion 14b of the proximal shaft 14. In some cases, the one or more lumens of the proximal shaft 14 may be configured to receive one or more elongated members in addition or as an alternative to the pull wire 18, where the one or more elongated members may be in the same lumen as the lumen receiving the pull wire 18 or in a lumen that is different than the lumen that receives the pull wire 18.

The proximal shaft 14 may be configured from a suitable material. In some cases, the proximal shaft 14 may be made from a polymer material, a metal material, a combination of polymer material and metal material, and/or one or more other suitable materials. Example polymer and metal materials, among other possible materials, are discussed below. In one example, the proximal shaft 14 may be formed from a polymer tube configured to traverse through a patient's vasculature. In another example, the proximal shaft 14 may be configured from a metal coil forming a tube or other structure with one or more lumens. The proximal shaft 14 may be a tubular member.

The distal shaft 16 may extend from the distal end portion 14b of the proximal shaft 14 and the pull wire 18 may extend along a length of the distal shaft 16 (e.g., inside and/or outside of the distal shaft 16). When the distal shaft 16 extends from the distal end portion 14b of the proximal shaft 14, the distal shaft 16 may extend distally from a distal terminal end 26 of the proximal shaft 14. Alternatively, the distal shaft 16 may extend distally from the handle 12.

The distal shaft 16 may be attached to or connected to the proximal shaft 14 in a suitable manner. For example, the distal shaft 16 may be attached or connected to the proximal shaft 14 via an adhesive connection, a threaded connection, a weave connection, a weld connection, a solder connection, and/or one or more other suitable connections.

The distal shaft 16 may have a proximal end portion 16a and a distal end portion 16b, with one or more lumens extending at least partially between the proximal end portion 16a and the distal end portion 16b. One or more lumens of the distal shaft 16 may extend an entire distance between the proximal end portion 16a and the distal end portion 16b of the distal shaft 16 and/or one or more lumens of the distal shaft 16 may extend for a portion of an entire distance between the proximal end portion 16a and the distal end portion 16b of the distal shaft 16. The distal shaft 16 may be a tubular member.

The distal shaft 16 may be formed from any suitable materials. In some cases, the distal shaft may be formed from a polymer material, a metal material, a metal material and a polymer material, and/or one or more other suitable materials. Example polymer and metal materials, among other possible materials, are discussed below. In one example, the distal shaft 16 may be a tubular member formed from an elongated polymer tube, an elongated metal tube, and/or one or more other tubular structures. The distal shaft 16 may be formed from a coiled wire. Example shafts formed from coiled wire are described in U.S. Ser. No. 15/094,188 filed on Apr. 8, 2016, which was published as U.S. 2016/0220265 A1, and is hereby incorporated in its entirety for all purposes. Alternatively or in addition, the distal shaft 16 may be formed in one or more other suitable manners.

When the distal shaft 16 is at least partially formed from a coiled wire, as depicted in the Figures, the wire forming the distal shaft 16 may be formed from any suitable material. For example, such wire may be formed from a polymer material, a metal material, a combination of metal material and polymer material, and/or one or more other suitable material. Example polymer and metal materials, among other possible materials, are discussed below. In one example, the wire forming the coils of the distal shaft 16 may be made out of a stainless steel (e.g., 304V stainless steel or other suitable stainless steel).

The wire forming coils of the distal shaft 16 may have a suitable diameter or thickness for facilitating navigation of the retraction device 10 through a patient's vasculature, interacting with tissue of the patient, and/or facilitating operation of the retraction device 10. In some cases, a diameter or thickness of the wire may be constant along a length of the wire or may vary to modify a stiffness of the distal shaft 16 along a length of the distal shaft 16. Example diameters of the wire forming coils of the distal shaft 16 may be in a range from about 0.017 mm or less to about 0.254 mm or greater. In one example, the wire forming coils of the distal shaft 16 may have a diameter of about 0.100 mm.

The distal shaft 16 may include one or more eyelets 28. The eyelets 28 may be formed at one or more intervals along a length of the distal shaft 16. In some cases, the eyelets 28 may be formed such that the pull wire 18 extending along the distal shaft 16 may extend through one or more of the eyelets 28. In one example, the pull wire 18 may be connected to the distal shaft 16, extend along an exterior surface of the distal shaft 16, and extend through one or more of the eyelets 28, as shown in FIG. 1. In the example, retraction of the pull wire 18 may cause the eyelets 28 to abut one another or move toward one another and form loops with the distal shaft 16 at locations between eyelets 28.

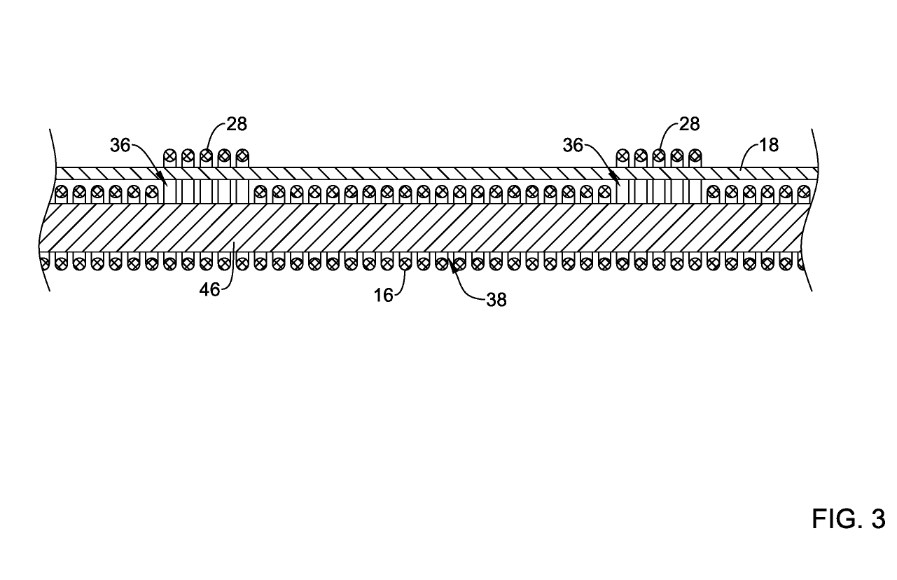

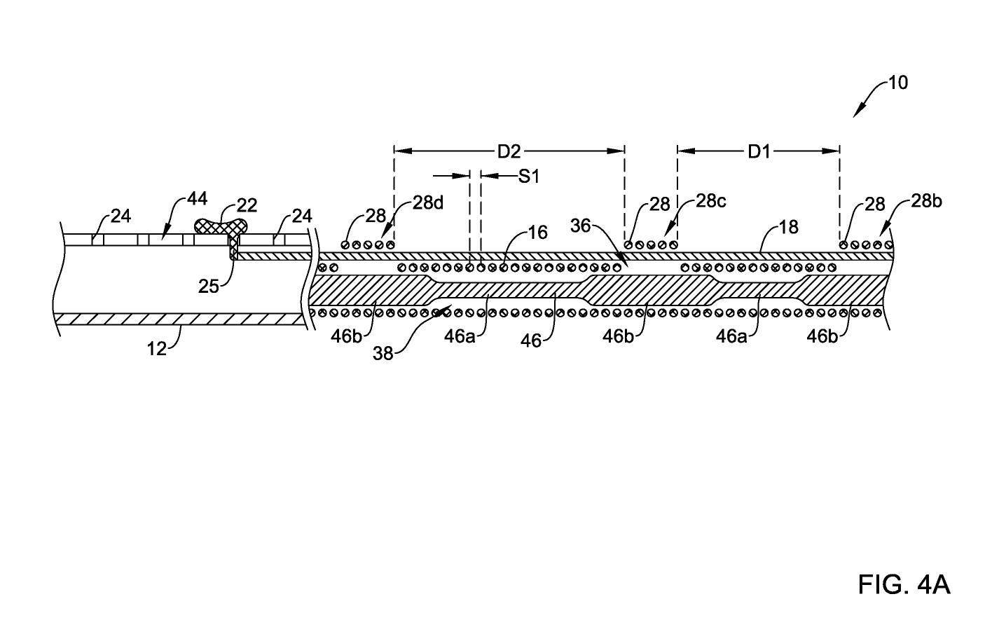

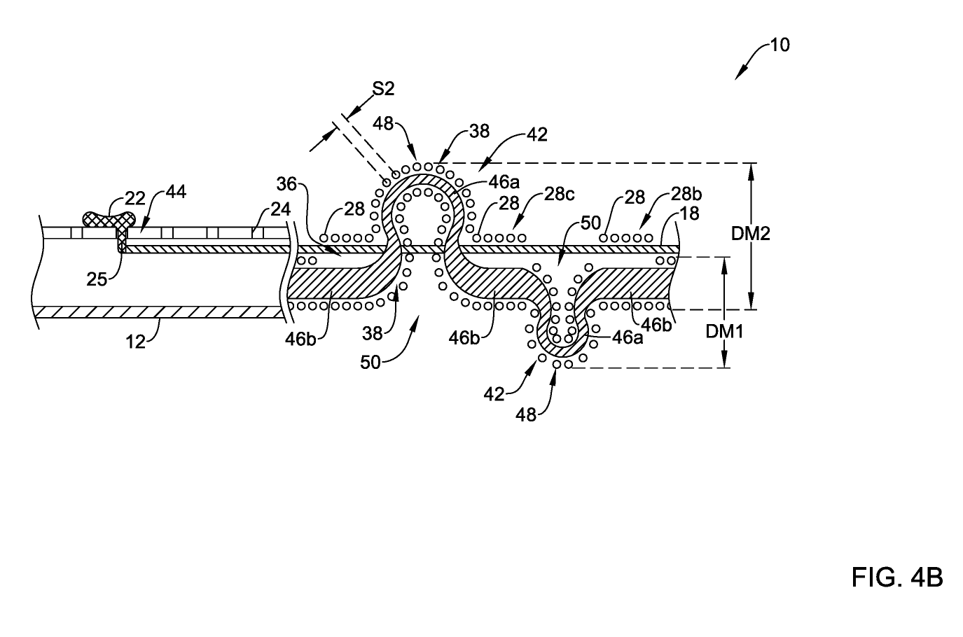

The eyelets 28 may be formed in one or more suitable manners such that the distal shaft 16 may have a greater outer diameter at a location of each eyelet 28 along the length of the distal shaft 16 than at locations along the length of the distal shaft 16 between eyelets 28 and/or between eyelets 28 and a terminal end of the distal shaft 16. Further, in some cases, the distal shaft 16 may include an eyelet lumen and a main lumen (e.g., see eyelet lumen 36 and main lumen 38 in FIGS. 3, 4A, and 4B), where the eyelet lumen may be located at an eyelet 28 and the main lumen may extend between the eyelets 28 and/or extend through one or more eyelets 28. As shown in FIGS. 3, 4A, and 4B, one or more of the eyelet lumens 36 may be in communication with the main lumen 38. Alternatively, or in addition, one or more of eyelet lumens 36 may be separated from the main lumen 38 by a layer of material, such that the one or more eyelet lumens 36 may be separated from and parallel to the main lumen 38.

The distal shaft 16 may include a suitable number of eyelets 28. In one example and as depicted in FIG. 1, the distal shaft 16 may include five eyelets 28 (e.g., starting from the distal end and moving proximal, the distal shaft 16 may include: a first eyelet 28a, a second eyelet 28b, a third eyelet 28c, a fourth eyelet 28d, and a fifth eyelet 28e) with each eyelet 28 spaced from a next eyelet 28 by a predetermined distance when the distal shaft is in an elongated state. The distal shaft 16 may include fewer than five (5) eyelets 28 or more than five (5) eyelets 28, as desired. The predetermined distance between the eyelets 28 may be a same distance between each eyelet 28 or a distance between a first set of adjacent eyelets 28 may be different than a distance between a second set of adjacent eyelets 28.

One example of spacing between eyelets 28 is depicted in FIG. 1. In FIG. 1, the first eyelet 28a may be spaced from a location 34 at which the pull wire 18 is attached or connected to the distal shaft 16 by a first distance, D1, the second eyelet 28b may be spaced from the first eyelet 28a by the first distance, D1, the third eyelet 28c may be spaced from the second eyelet 28b by the first stance, D1, the fourth eyelet 28d may be spaced from the third eyelet 28c by a second distance, D2, the fifth eyelet 28e may be spaced from the fourth eyelet 28d by the second distance, D2, and the distal terminal end 26 of the proximal shaft 14 and/or a proximal terminal end 40 of the distal shaft 16 may be spaced from the fifth eyelet 28d by the second distance, D2, where the second distance, D2, may be greater than the first distance, D1. However, the distance between the eyelets 28 may have different spacing relative to spacing between other eyelets 28 than what is described with respect to the example shown in FIG. 1.

The distances between eyelets 28 may be any suitable distances. In some cases, the distances between eyelets 28 may be configured to obtain one or more predetermined diameters of loops when the pull wire 18 has been retracted (e.g., the loops are discussed in greater detail below). In some cases, the distances between eyelets 28 may be in a range from about one (1) mm or less to about forty (40) mm or greater, from about five (5) mm to about thirty-five (35) mm, from about ten (10) mm to about thirty (30) mm, and/or within one or more other suitable ranges. In one example, the distances between eyelets 28 may be in a range from about ten (10) mm to about twenty-five (25) mm. In the example discussed above with the first distance, D1, and the second distance, D2, the first distance, D1, may be or may be about sixteen (16) mm and the second distance, D2, may be or may be about twenty-one (21) mm.

Distances between eyelets 28 may be determined based on an intended use of the retraction device 10, but this is not required. For example, a retraction device 10 intended to be used in cerebral vessels to address stroke issues may have distances between eyelets 28 in a range from about one (1) mm to about ten (10) mm, a retraction device 10 intended to be used in a vena cava may have distances between eyelets 28 in a range from about twenty (20) mm to about forty (40) mm, a retraction device 10 intended to be used in a femoral or popliteal vessel may have distances between eyelets 28 in a range from about fifteen (15) mm to about nineteen (19) mm, a retraction device 10 indented to be used in pulmonary vessels to treat pulmonary embolisms may have distances between eyelets 28 in a range from about ten (10) mm to about forty (40) mm. Other ranges for the above uses and/or other uses of the retraction device 10 may be utilized as desired or is suitable for the purpose.

The distal shaft 16 may have a distal tip portion 30 adjacent the distal end portion 16b of the distal shaft 16. In one example, the distal tip portion 30 may extend from a terminal distal end 32 of the distal shaft 16 to the location 34 at which the pull wire 18 may be attached to or connected to the distal shaft 16, as shown in FIG. 1. In some cases, the distal tip portion 30 may be located distal of a distal most eyelet 28.

The distal tip portion 30 may have any suitable length. Example lengths of the distal tip portion 30 may include lengths from about one (1) millimeter (mm) or less and about thirty (30) mm or greater, from about five (5) mm and about twenty-five (25) mm, and/or from about ten (10) mm and about twenty (20) mm. In one example, for a retraction device 10 configured to remove thrombi (e.g., acute or sub-acute thrombi) or other objects from a patient's vessel having a diameter from about ten (10) mm to about eighteen (18) mm, the distal tip portion 30 may have a length of about twelve (12) mm. In such an example and/or other examples, the length of the distal tip portion 30 may be configured to facilitate centering one or more loops formed in the distal shaft 16 within a patient's vessel as other lengths of the distal tip portion 30 may bias the formed loops to one side of the patient's vessel. The distal tip portion may be configured for other purposes including, but not limited to, for navigation through a patient's vessels, for passing through an obstruction in a patient's vessels, for contact with patient tissue, and/or for one or more other purposes, as desired.

The pull wire 18 may be attached to or connected to the distal shaft 16 at the location 34 (e.g., distal of the distal-most eyelet 28, as shown in FIG. 1, or other suitable location) through a suitable attaching or connection technique. Example suitable techniques include, but are not limited to, connections via, adhesives, welding, soldering, fusion bonding, and/or one or more other suitable techniques.

The pull wire 18 may be formed from any suitable material. For example, the pull wire 18 may be formed from a polymer material, a metal material, a combination of metal material and polymer material, and/or one or more other suitable material. Example polymer and metal materials, among other possible materials, are discussed below. In one example, the pull wire 18 may be made out of a nitinol material.

The pull wire 18 may have a suitable diameter for facilitating navigation of the retraction device 10 through a patient's vasculature and retraction through the eyelets 28. For example, the diameter of the pull wire 18 may be in a range from about 0.050 mm to about 0.210 mm. In one example, the pull wire 18 may have a diameter of about 0.100 mm.

The pull wire 18 may have one or more tapered profiles along its length. In one example, the pull wire 18 may have a proximal diameter of about 0.210 mm, have a first taper proximal of the proximal-most eyelet 28 (e.g., the eyelet 28e in the example depicted in FIG. 1) to an intermediate diameter of about 0.100 mm, and have a second taper distal of the distal-most eyelet 28 (e.g., the eyelet 28a in the example depicted in FIG. 1) and/or distal of the location 34 at which the pull wire 18 is attached to the distal tip portion 30 to a distal diameter of about 0.050 mm. Other suitable taper profiles may be utilized as desired.

FIG. 2 depicts a schematic top view of the retraction device 10 with the adjustment member 22 in a fully retracted position and the loops 42 formed in, and forming a withdrawal member of, the distal shaft 16. The adjustment member 22 may be adjustable along an elongated slide path 44 (e.g., a channel) extending through a portion of a housing of the handle 12 and as the adjustment member 22 is in communication with the pull wire 18, when the adjustment member 22 is adjusted the pull wire 18 may be retracted to form the loops 42.

FIG. 3 depicts a schematic view depicting a cross-section of a portion of the retraction device 10 extending along the distal shaft 16, with the pull wire 18 (e.g., a first elongated member) and a core wire 46 (e.g., a second elongated member) extending along the distal shaft 16. The core wire 46 may be configured to provide a desired stiffness of the loops 42 that facilitates retracting a thrombi or other object from a vessel or cavity while still being able to retract the loops 42 through the vessel, cavity, catheter, sheath, etc.

The core wire 46 may have a constant stiffness along its length or a variable stiffness along its length. In one example, the core wire 46 may have a first stiffness at locations spaced between the eyelets 28 and a second stiffness at locations of or adjacent to the eyelets 28, where the first stiffness is a less stiff stiffness than the second stiffness. In the example, the retraction device may have a less stiff portion at a crown (e.g., a crown 48, as depicted in FIG. 4B) of the loops 42 formed between the eyelets 28 than at a base (e.g., a base 50, as depicted in FIG. 4B) of the loops 42 formed between the eyelets 28, where the base may include eyelets 28 and/or portions between the loops 42. Alternatively or in addition, the core wire 46 may be configured to have a different stiffness for each of one or more of the loops 42 to be formed when the pull wire 18 is retracted. The differences in stiffness of the core wire 46 may be due to differences in material of the core wire 46 at different locations along the core wire 46, differences in diameter or thickness of the core wire 46, cuts in the core wire 46 at different locations along the core wire 46, and/or due to other differences in the core wire along its lengths. Such variable stiffness in the core wire 46 may facilitate forming loops that mitigate damage to a vessel or cavity wall when the loops are formed, while still facilitating withdrawal of thrombi or other objects due to the stiffness at the base of the loops.

The core wire 46 may be formed from any suitable material. For example, the core wire 46 may be formed from a polymer material, a metal material, a combination of metal material and polymer material, and/or one or more other suitable material. Example polymer and metal materials, among other possible materials, are discussed below. In one example, the core wire 46 may be made out of a nitinol material.

The core wire 46 may have a suitable diameter or thickness for facilitating navigation of the retraction device 10 through a patient's vasculature and, in some cases, for providing a desired stiffness to the distal shaft 16 to prevent or mitigate kinking in the distal shaft and facilitating withdrawal of thrombi and/or other objects from a vessel or cavity. For example, the diameter of the core wire 46 may be in a range from about 0.050 mm to about 0.360 mm. In one example, the core wire 46 may have a diameter of about 0.150 mm. In another example, the core wire 46 may have a diameter of about 0.203 mm.

As depicted in FIG. 3, the core wire 46 may have a constant diameter along its length or at least a portion of its length extending along the distal shaft 16. Alternatively, as depicted in FIGS. 4A and 4B, the core wire 46 may have a diameter or thickness that varies along its length or at least a portion of its length extending along the distal shaft 16.

The core wire 46 may have a variable diameter or thickness, as depicted in FIGS. 4A and 4B, but this is not required and the core wire 46 may have a constant diameter along the length of the portion of the core wire extending along the distal shaft 16. To facilitate the crown 48 of the loops 42 being less stiff than the base 50 of the loops 42, one or more the portions (e.g., portions 46a) of the core wire 46 extending through the distal shaft 16 between eyelets 28 may have a reduced diameter when compared to one or more portions (e.g., portions 46b) of the core wire 46 extending through the eyelets 28. Such a configuration may provide a stiff base 50 of the loops 42 and a soft or less stiff crown 48 (e.g., relative to a stiffness at the base 50) and/or edges of the loops 42 to facilitate removing a center lumen thrombi or other object from a vessel or cavity in an efficient manner while providing minimal or mitigated damage to the walls of the vessel or cavity. Alternatively or in addition, the core wire 46 may be configured to have a different diameter or thickness for each of one or more of the loops 42 to be formed when the pull wire 18 is retracted. Different diameters and/or thicknesses of the core wire 46 at the various loops 42 may facilitate using the retraction device 10 in vessels or cavities with diameters that may change abruptly along their length.

FIGS. 4A and 4B depict a schematic view of a cross-section of a portion of the retraction device 10 extending along the distal shaft 16 and the handle 12. FIG. 4A depicts the retraction device 10 in a partially retracted position, as evidenced by the adjustment member 22 engaging a restriction 24 between a proximal-most depicted restriction 24 and a distal-most depicted restriction 24. FIG. 4B depicts the retraction device 10 in a different further partially retracted position, as evidenced by the adjustment member 22 engaging a restriction 24 further proximal of the restriction 24 engaged in FIG. 4A, but still between the proximal-most depicted restriction 24 and the distal-most depicted restriction 24. More specifically and with respect to the example discussed above, where the distal shaft 16 may have five (5) eyelets 28, the eyelets 28 depicted in FIGS. 4A and 4B may be eyelets 28b, 28c, and 28d, where a distance between eyelets 28b and 28c may be the first distance, D1, and a distance between eyelets 28c and 28d may be the second distance, D2.

As depicted in FIGS. 4A and 4B, the adjustment member 22 may extend through the slide path 44 extending through a portion of the handle 12 and may engage the pull wire 18. In response to movement of the adjustment member 22 as depicted in advancing from FIG. 4A to FIG. 4B, the pull wire 18 may retract and pull the eyelets 28 close to adjacent eyelets (e.g., the second eyelet 28b is pulled close to the third eyelet 28c and the third eyelet 28c is pulled close to the fourth eyelet 28d) to form loops 42, as shown in FIG. 4B.

When the loops 42 are formed and the distal shaft 16 is formed from a coiled wire, as depicted in FIG. 4B, adjacent coils of the portion of the coiled wire forming the loops 42 may separate from one another. This is evident when comparing a first spacing, S1, between coils of the distal shaft 16 depicted in FIG. 4A prior to the pull wire 18 being retracted to form loops 42 to a second spacing, S2, between coils of the distal shaft 16 depicted in FIG. 4B after the pull wire 18 has been retracted to form the loop 42, where the second spacing, S2, is greater than the first spacing, S1. Such greater spacing between coils forming the loop 42 may facilitate engaging thrombi and/or other objects in a vessel or cavity to withdraw the thrombi and/or other objects from the vessel or cavity.

As referred to above, a distance between eyelets 28 when the distal shaft 16 is an elongated state may be proportional to an outer diameter of the loops 42 when the distal shaft 16 is in a retracted state. For example, as the first distance, D1, between the second eyelet 28b and the third eyelet 28c is less than the second distance, D2, between the third eyelet 28c and the fourth eyelet 28d, a first diameter, DM1, of a loop 42 formed between the second eyelet 28b and the third eyelet 28c may be less than a second diameter, DM2, of a loop 42 formed between the third eyelet 28c and the fourth eyelet 28d.

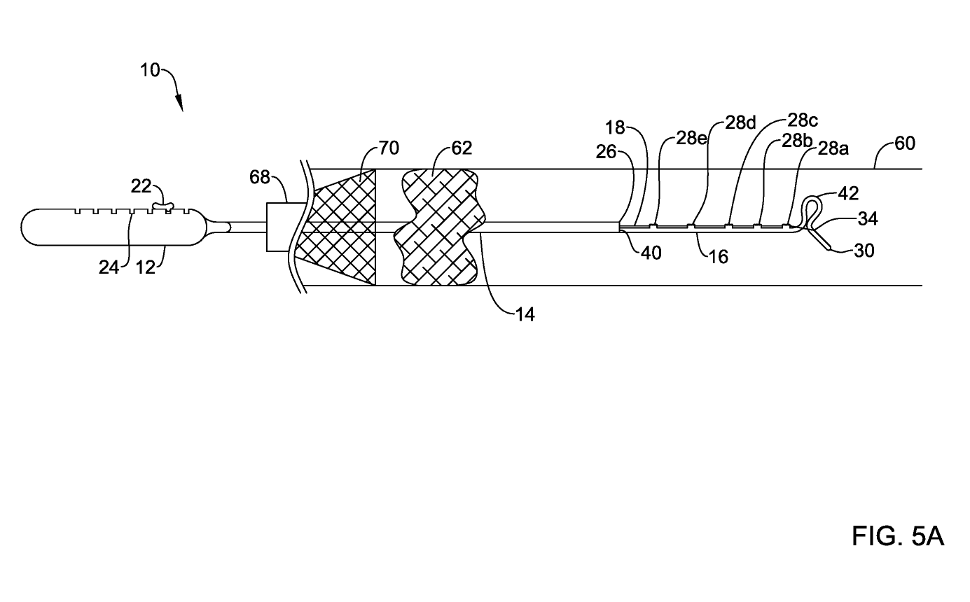

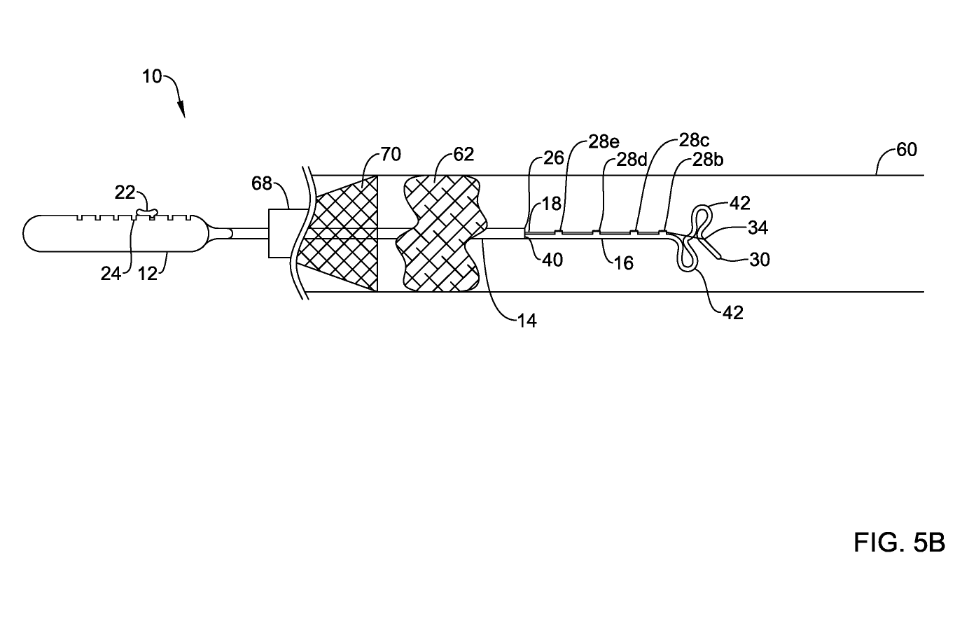

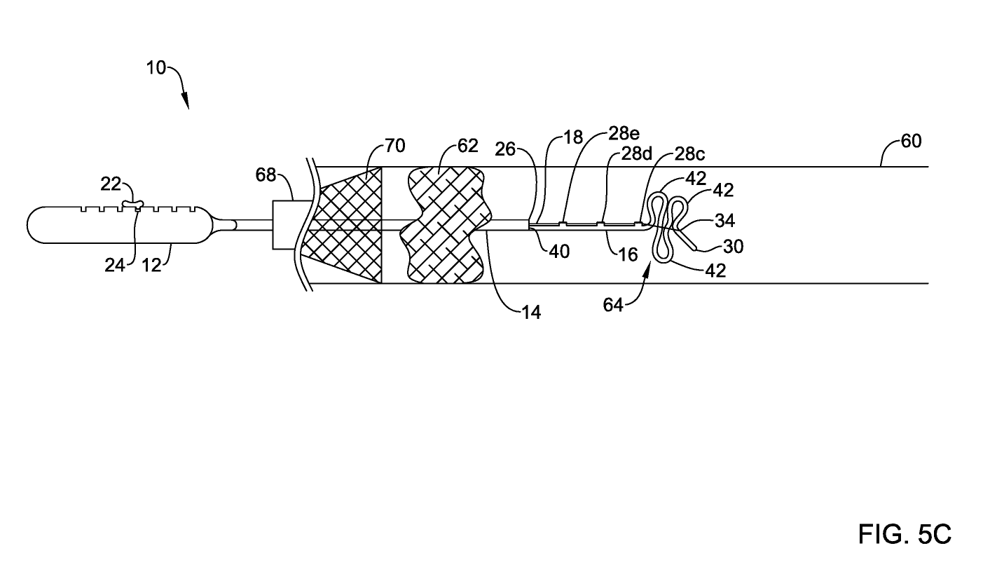

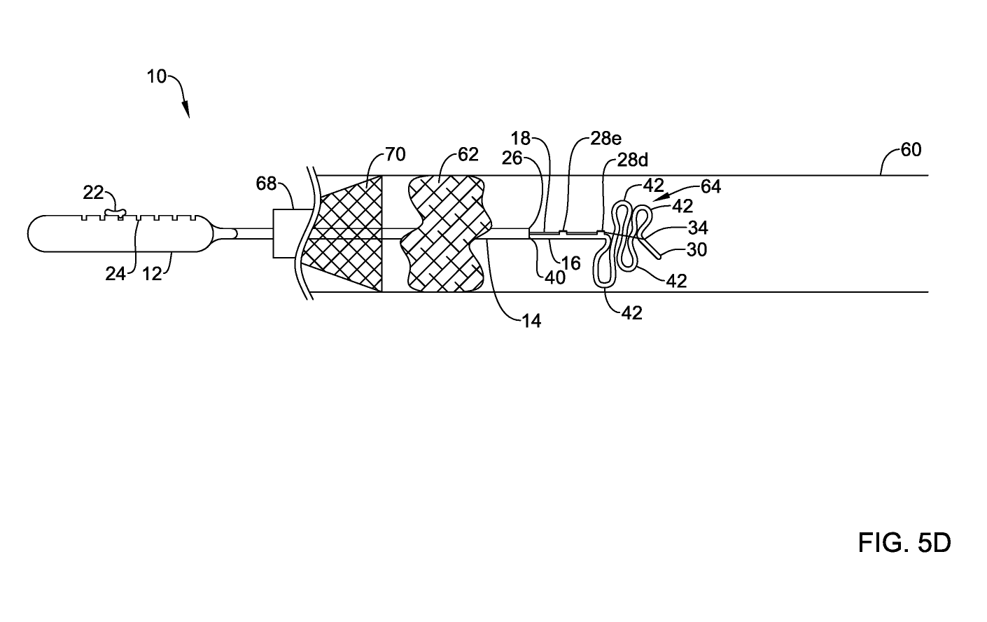

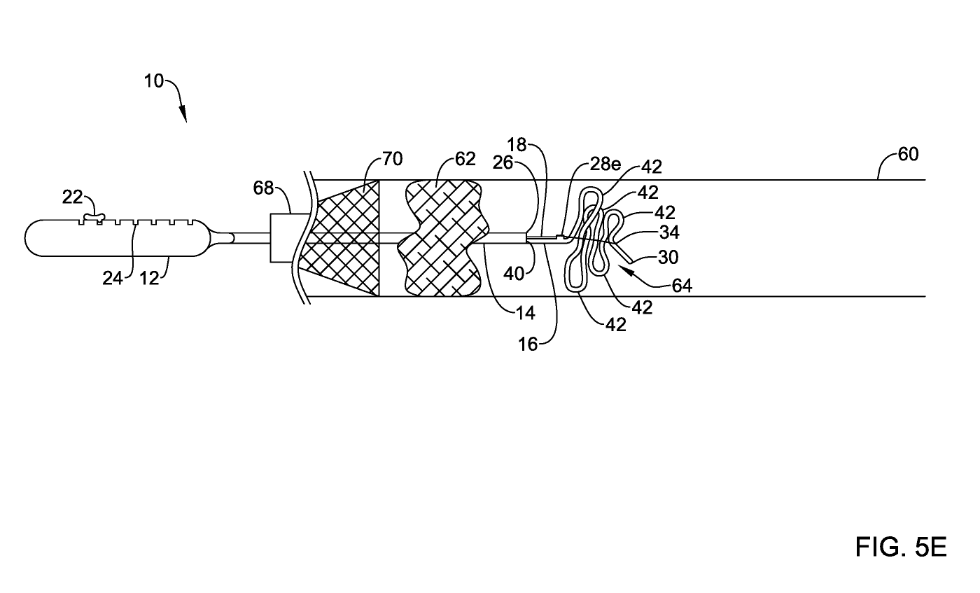

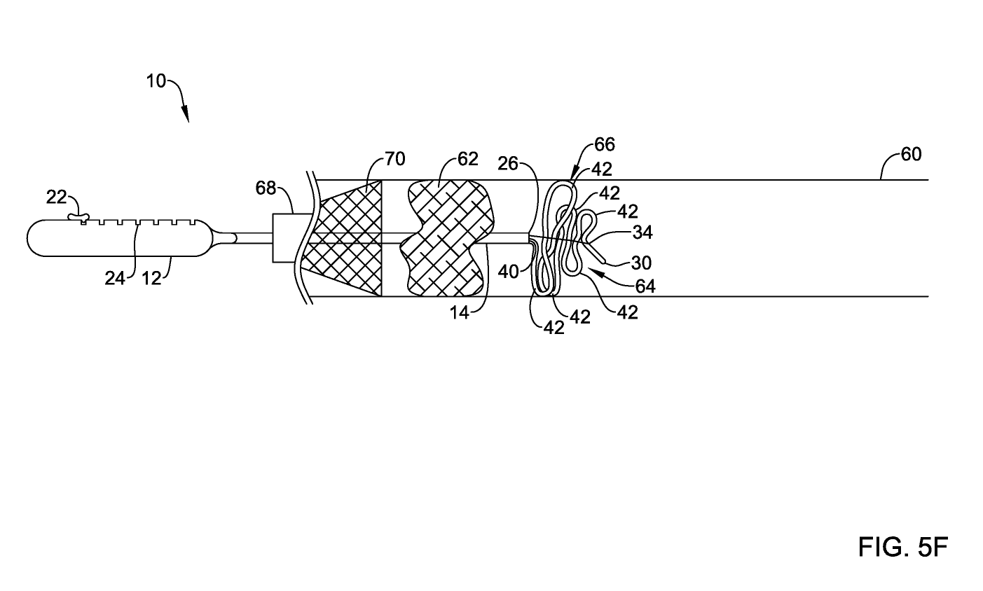

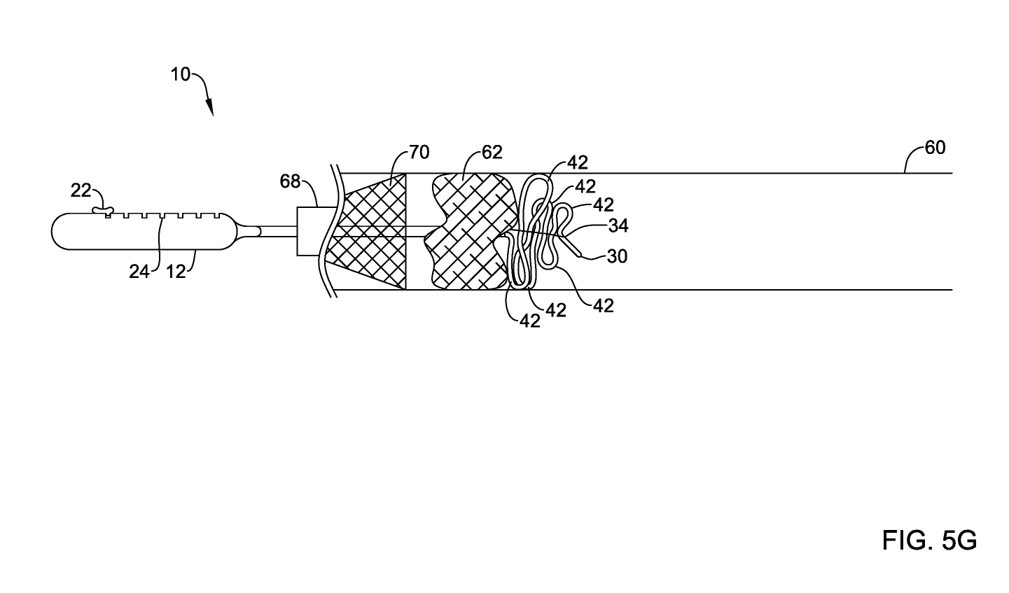

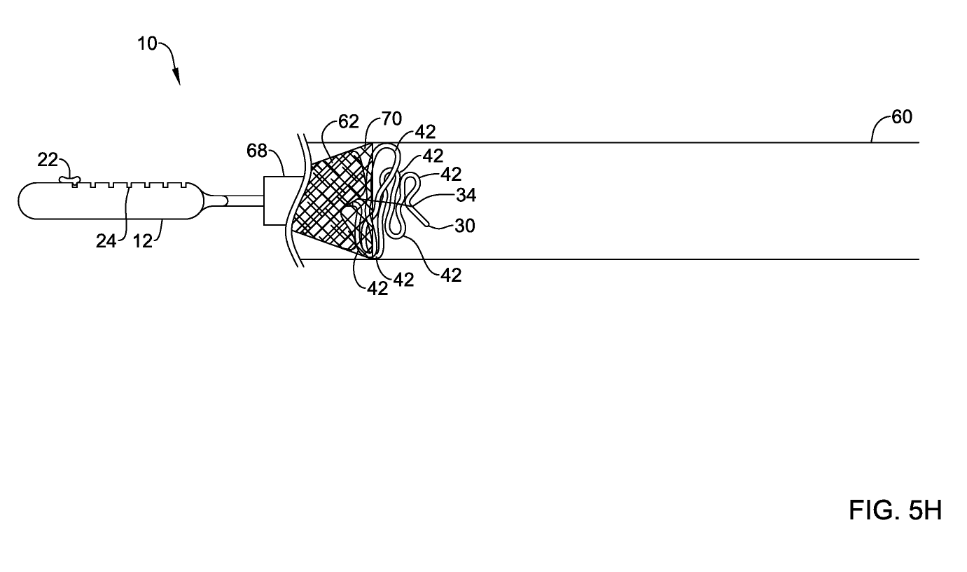

FIGS. 5A through 5H depict an example retraction device 10 in use within a vessel 60 (e.g., the vessel 60 is shown in cross-section) to withdraw an obstruction 62 (e.g., thrombus or other object) from the vessel 60. FIGS. 5A-5C depict the formation of a first set or section 64 of the loops 42 each having an equal or substantially equal first diameter. FIGS. 5D-5F depict the formation of a second set or section 66 of the loops 42 each having an equal or substantially equal second diameter. FIGS. 5G and 5H depict a withdrawal and capture of the obstruction 62.

Turning to FIG. 5A, a sheath 68 may be disposed within the vessel 60 of a patient and a catheter (not shown) having a capture filter 70 may be inserted through the sheath 68 and into the vessel 60. The sheath 68, the capture filter 70, the catheter, the retraction device 10, and/or other suitable medical devices may be part of a retraction device system. The capture filter 70 may be a fabric, mesh, basket, sock, and/or other configuration configured to receive the obstruction 62 when it is pulled into the capture filter 70 with the loops 42. In some cases, the capture filter 70 may be omitted and/or a second capture filter may be included distal of the formed loops 42 to capture portions of the obstruction 62 that flow downstream of the loops 42. The proximal shaft 14 and the distal shaft 16 may be advanced through the sheath 68, past the capture filter 70, and past the obstruction 62. In some cases, the distal terminal end 26 of the proximal shaft 14 may be inserted past the obstruction 62 or past at least part of the obstruction 62 to facilitate formation of the loops 42 distal of the obstruction 62. As depicted in FIG. 5A, a first loop 42, having a first diameter, has been formed between the location 34 at which the pull wire 18 is attached or connected to the distal shaft 16 and a distal-most eyelet 28 in response to movement of the adjustment member 22 such that it is engaging a second restriction 24 (e.g., where the restrictions 24 are counted from a distal end to a proximal end of the restrictions) and retracts the pull wire 18 a first predetermined distance.

FIGS. 5B and 5C depict substantially the same representation as that of FIG. 5A, but with the additional loops 42 formed in the retraction device 10. FIG. 5B depicts a second loop 42, having the first diameter, formed in the retraction device 10, where the second loop 42 may be formed between the first eyelet 28a and the second eyelet 28b in response to movement of the adjustment member 22 such that it is engaging a third restriction 24 and retracts the pull wire 18 the first predetermined distance. FIG. 5C depicts a third loop 42, having the first diameter, formed in the retraction device 10, where the third loop 42 may be formed between the second eyelet 28b and the third eyelet 28c in response to movement of the adjustment member 22 such that it is engaging a fourth restriction 24 and retracts the pull wire 18 the first predetermined distance. In the example of FIGS. 5A-5H, once the third loop 42 has been formed, the first set or section 64 of loops 42 has been formed. Although the first set or section 64 of the loops 42 is depicted with three (3) loops 42, the first set or section 64 of the loops 42 may have fewer than three (3) loops 42 or more than three (3) loops 42.

FIGS. 5D-5F depict substantially the same representation as that of FIGS. 5A-5C, but depict the formation of loops 42 of the second set or section 66 of the loops 42. FIG. 5D depicts a fourth loop 42, having a second diameter, formed in the retraction device 10, where the fourth loop 42 may be formed between the third eyelet 28c and the fourth eyelet 28d in response to movement of the adjustment member 22 such that it is engaging a fifth restriction 24 and retracts the pull wire 18 a second predetermined distance. FIG. 5E depicts a fifth loop 42, having the second diameter, formed in the retraction device 10, where the fifth loop 42 may be formed between the fourth eyelet 28d and the fifth eyelet 28e in response to movement of the adjustment member 22 such that it is engaging a sixth restriction 24 and retracts the pull wire 18 the second predetermined distance. FIG. 5F depicts a sixth loop 42 formed in the retraction device 10, where the sixth loop 42 may be formed between the fifth eyelet 28e and the distal terminal end 26 of the proximal shaft 14 in response to movement of the adjustment member 22 such that it is engaging a sixth restriction 24 and retracts the pull wire 18 a predetermined distance. In some cases, the sixth loop 42 may be a partial loop having a smaller or at least a different diameter than the second diameter and the adjustment member 22 may be adjusted such that the pull wire 18 may be retracted a predetermined distance that is less than or at least different than the second predetermined distance, which may facilitate ensuring the formed loops 42 are perpendicular or substantially perpendicular to a flow of fluid through the vessel 60. In such cases, a last restriction 24 in the handle 12 may be spaced from an immediately prior restriction 24 to provide an indication as the adjustment member 22 is moved that the pull wire 18 has been retracted the predetermined distance that is less than or at least different than the second predetermined distance. In the example of FIGS. 5A-5H, once the sixth loop 42 has been formed, the first set or section 64 of the loops 42 and the second section 66 of the loops 42 have been formed to create the withdrawal member in the retraction device 10. Although the second set or section 66 of the loops 42 is depicted with three (3) loops 42, the second set or section 66 of loops 42 may have fewer than three (3) loops 42 or more than three (3) loops 42.

The loops 42 of the first set or section 64 of the loops 42 may form a loop structure having a diameter and the loops 42 of the second set or section 66 of the loops 42 may form a loop structure having a second diameter. The diameter of the first set or section 64 of the loops 42 may be configured to be less than the diameter of the second set or section 66 of the loops 42. Such a configuration may facilitate centering the formed loops 42 within the vessel 60 and/or maintaining the formed loops in an orientation that is generally perpendicular to a flow of fluid through the vessel 60. In some cases, the diameter of the loop structure of the first set or section 64 of the loops 42 may be configured such that the crowns of the loops 42 are spaced from a wall of the vessel 60 and one or more of the crowns of the loops 42 of the second set or section 64 of the loops 42 are adjacent to or touching the wall of the vessel 60 when both of the first set or section 64 of loops 42 and the second set or section 66 of loops 42 are formed in the retraction device 10.

Once the first and second sets or sections 64, 66 of the loops 42 have been formed in the retraction device 10, the retraction device 10 may be withdrawn from the vessel such that the formed loops 42 engage the obstruction 62, as depicted in FIG. 5G. Further, the retraction device 10 may be withdrawn until the loops 42 reach and/or are within a distal end or opening of the capture filter 70 and at least part of the obstruction 62 is sandwiched between the capture filter 70 and the loops 42, as depicted in FIG. 5H. In some cases, the loops 42 may act as a cap on the capture filter 70. Once, the loops 42 reach the distal end or opening of the capture filter 70, the capture filter 70 and loops 42 may be withdrawn or retracted from the vessel 60. If a portion of the obstruction 62 remains within the vessel 60, the technique described above with respect to FIGS. 5A-5H may be repeated.

Although a technique for using the retraction device 10 is described herein, other techniques of using the retraction device are contemplated. In one example of an alternative technique, among others, the capture filter 70 may be deployed distal of the obstruction 62, the loops 42 may be formed proximal of the obstruction 62, the loops 42 may be used to push the obstruction 62 distally into the capture filter 70, and then, the capture filter 70, the obstruction 62, and the loops 42 may be withdrawn from the vessel 60. Additionally, although not necessarily depicted in the Figures, the techniques described herein may include one or more steps other than those steps described herein and/or the described steps may be performed in one or more other orders, as desired unless expressly indicated otherwise. Further, it is contemplated that the retraction device 10 may be utilized to treat deep vein thrombosis, pulmonary embolisms, and/or other conditions in which it may be desirable to retrieve an object from a vessel or cavity of a patient.

Although specific materials may be discussed above for the retraction device 10, components of the retraction device 10 may include suitable materials commonly associated with medical devices. For simplicity purposes, the following discussion makes reference to the retraction device 10. However, this is not intended to limit the devices and methods described herein, as the discussion may be applied to other similar systems and/or components of systems or devices disclosed herein.

Components of the retraction device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the polymer can be blended with a liquid crystal polymer (LCP). For example, the blend can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the components of the retraction device 10 (e.g., at the distal tip portion 30 or other suitable locations) may be doped with, made of, or otherwise include a radiopaque material. Radiopaque materials are understood to be materials capable of producing a relatively bright image on a fluoroscopy screen or another imaging technique during a medical procedure. This relatively bright image aids the user of the retraction device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the retraction device 10 to achieve the same result.

In some embodiments, a degree of Magnetic Resonance Imaging (MRI) compatibility is imparted into the retraction device 10. For example, the retraction device 10, or portions or components thereof, may be made of a material that does not substantially distort the image and create substantial artifacts (i.e., gaps in the image). Certain ferromagnetic materials, for example, may not be suitable because they may create artifacts in an MRI image. The retraction device 10, or portions thereof, may also include and/or be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A retraction device comprising:
a tubular member having a first end portion, a second end portion, and a lumen extending from the first end portion to the second end portion;
a plurality of eyelets spaced along the tubular member;
a first elongated member extending through a first eyelet of the plurality of eyelets;
a second elongated member extending through the lumen of the tubular member; and
wherein a stiffness of the second elongated member varies along a length of the second elongated member.

2. The retraction device of claim 1, wherein the second elongated member has a first stiffness at a location adjacent the first eyelet that is different than a second stiffness at a location between the first eyelet and a second eyelet of the plurality of eyelets.

3. The retraction device of claim 2, wherein the second stiffness is less than the first stiffness.

4. The retraction device of claim 2, wherein the second elongated member has a first thickness at the location adjacent the first eyelet that is greater than a second thickness at the location between the first eyelet and the second eyelet.

5. The retraction device of claim 1, wherein first elongated member is connected to the tubular member at a location distal of a distal-most eyelet of the plurality of eyelets.

6. The retraction device of claim 1, wherein retraction of the first elongated member is configured to cause the tubular member to form a loop between the first eyelet and a second eyelet of the plurality of eyelets.

7. The retraction device of claim 6, wherein:
the loop has a base at an end portion of the loop adjacent the first eyelet and the second eyelet and a crown at a location of the loop opposite the base; and
the second elongated member has a first stiffness adjacent the base of the loop and the second elongated member has a second stiffness adjacent the crown of the loop.

8. The retraction device of claim 1, wherein:
a second eyelet of the plurality of eyelets is spaced from the first eyelet a first distance;
a third eyelet of the plurality of eyelets is spaced from the second eyelet by the first distance;
a fourth eyelet of the plurality of eyelets is spaced from the third eyelet a second distance;
a fifth eyelet of the plurality of eyelets is spaced from the fourth eyelet by the second distance; and
the first distance is less than the second distance.

9. The retraction device of claim 8, wherein:
the first elongated member is connected to the tubular member at a location distal of the first eyelet; and
retraction of the first elongated member causes:
a first loop to form between the location distal of the first eyelet and the first eyelet;
a second loop to form between the first eyelet and the second eyelet;
a third loop to form between the second eyelet and the third eyelet;
a fourth loop to form between the third eyelet and the fourth eyelet; and
a fifth loop to form between the fourth eyelet and the fifth eyelet.

10. The retraction device of claim 1, further comprising:
a shaft extending proximally from the first end portion of the tubular member; and
wherein retraction of the first elongated member causes a loop to form between a proximal-most eyelet of the plurality of eyelets and a distal end portion of the shaft.

11. The retraction device of claim 1, further comprising:
a handle;
an adjustment member adjacent the handle; and
wherein adjusting the adjustment member retracts the first elongated member and forms a loop in the tubular member between the first eyelet and a second eyelet of the plurality of eyelets.

12. The retraction device of claim 11, wherein the handle includes a plurality of adjustment restrictions to limit adjustment of the adjustment member at predetermined intervals.

13. A retraction device comprising:
a shaft having a first end portion and a second end portion;
an elongated member extending along the shaft and connected to the second end portion of the shaft;
wherein:
retraction of the elongated member toward the first end portion of the shaft results in forming a first section of a plurality of loops in the shaft and a second section of a plurality of loops in the shaft; and
two or more of the plurality of loops of the first section have a first diameter that is less than a second diameter of two or more of the plurality of loops of the second section.

14. The retraction device of claim 13, wherein the first section is distal of the second section.

15. The retraction device of claim 13, wherein the shaft is formed from a coiled wire.

16. The retraction device of claim 13, wherein the elongated member is a first elongated member and the retraction device further comprises:
a second elongated member extending along the shaft;
wherein the second elongated member has a varying stiffness along at least one loop of the first section or the second section with a first stiffness adjacent a base of the at least one loop and a second stiffness adjacent a crown of the at least one loop, the second stiffness is less than the first stiffness.

17. The retraction device of claim 13, wherein the elongated member is a first elongated member and the retraction device further comprises:
a second elongated member extending along the shaft;
wherein the second elongated member has a varying stiffness along a length of the second elongated member with a first stiffness along a first loop of the first section or the second section and a second stiffness along a second loop of the first section or the second section, the second stiffness is less than the first stiffness.

18. The retraction device of claim 13, further comprising:
eyelets spaced along the shaft; and
wherein a portion of the shaft extending between consecutive eyelets forms a loop of the first section or the second section.

19. A method of using a retraction device, the method comprising:
retracting a pull wire having a distal end portion connected to a distal end portion of a shaft;
forming two or more loops with the shaft of a first set of loops in response to retracting the pull wire;
forming two or more loops with the shaft of a second set of loops in response to retracting the pull wire; and
wherein the shaft is configured such that at least two loops of the first set of loops each has a first diameter and at least two loops of the second set of loops each has a second diameter different than the first diameter.

20. The method of claim 19, further comprising:
forming a loop with the shaft between a proximal-most eyelet of the shaft and a proximal terminal end of the shaft; and
wherein the pull wire extends through the proximal-most eyelet of the shaft.
